(19) United States
(12) Patent Application Publication
(10) Pub. No.: US 2019/0376117 A1
(43) Pub. Date: Dec. 12, 2019

(54) COMPOSITION FOR DIAGNOSING CANCER USING PKA ACTIVITY AND INFORMATION PROVIDING METHOD FOR DIAGNOSING METASTASIS

(71) Applicants: AMOGREENTECH CO., LTD., Gyeonggi-do (KR); KANGWON NATIONAL UNIVERSITY UNIVERSITY-INDUSTRY COOPERATION FOUNDATION, Gyeonggi-do (KR)

(72) Inventors: Kwon-Soo Ha, Gangwon-do (KR); Deok-Hoon KONG, Gangwon-do (KR)

(21) Appl. No.: 16/542,924

(22) Filed: Aug. 16, 2019

Related U.S. Application Data
(62) Division of application No. 15/123,840, filed on Sep. 6, 2016.

(30) Foreign Application Priority Data
Mar. 6, 2014 (KR) 10-2014-0026755

(51) Int. Cl. C12Q 1/48; C12N 9/12

(52) U.S. Cl. CPC C12Q 1/485; C12N 9/1205

(57) ABSTRACT

Disclosed is a composition for diagnosing cancer, including an agent for measuring PKA (Protein Kinase A) activity.

### CROSS-REFERENCE TO RELATED APPLICATIONS

This application is a divisional of application Ser. No. 15/123,840, filed Sep. 6, 2016, which is a national phase application from, and claims priority to, International Patent Application No. PCT/KR2014/004282, filed May 13, 2014, which in turn claims priority to Korean Patent Application No. 10-2014-0026755, filed Mar. 6, 2014, all of which are incorporated herein by reference in their entirety.

### TECHNICAL FIELD

The present invention relates to a composition for diagnosing cancer, including an agent for measuring PKA (Protein Kinase A) activity.

### BACKGROUND ART

Cancer is the most common cause of disease-related death in the world. Hence, the early detection of cancer is very important.

With regard thereto, serological cancer biomarkers are widely useful in cancer diagnosis based on antigen determination, and may include, for example, a-fetoprotein (AFP) for hepatic cancer diagnosis, prostate-specific antigen (PSA) for prostate cancer diagnosis, carcinoembryonic antigen (CEA) for colorectal cancer diagnosis, cancer antigen CA 15-3 for breast cancer diagnosis, cancer antigen CA19-9 for gastric cancer diagnosis, and cancer antigen CA125 for ovarian cancer diagnosis. Furthermore, numerous potential protein markers, including metabolic enzymes, proteins associated with the cytoskeleton, and tumor suppressors, are reported to be useful in the diagnosis of cancer. However, these antigens are insufficient in sensitivity and/or specificity for cancer diagnosis. Therefore, novel and ideal cancer biomarkers and improved diagnosis methods are required in order to improve cancer diagnosis.

Meanwhile, cyclic AMP (cAMP)-dependent PKA (Protein Kinase A) is the most important enzyme for post-transcriptional modification, and plays an important role in a variety of biological procedures, such as cell proliferation, metabolism, gene induction, angiogenesis, the regulation of ion channels, and apoptosis. PKA, including Type I and Type II, is mainly an intracellular enzyme, namely a tetrameric enzyme comprising two regulatory units and two common catalytic subunits, and is separated into an R dimer and two free C-subunits by cAMP. No attempts have been made to date to utilize PKA or PKA activity as a marker for cancer diagnosis, and through a more sensitive approach such as an array-based assay, there is a need to evaluate whether sPKA activity and/or autoantibodies are able to serve as serological biomarkers for cancer diagnosis.

Also, PKA activity is often measured using radioactive isotope-labeled ATP, but typical methods are known to have deficiencies such as the risk of radiation, complications, excessive time consumption and the like. With the goal of overcoming these deficiencies, alternative non-radioactive methods based on fluorescence, luminescent nanoparticles and a quartz crystal microbalance have been proposed. In the fluorescence detection methods, molecular probes such as biotinylated phosphate-specific ligands based on a Zn²⁺ complex and pro-Q diamond dyes are used. Various types of nanoparticles, such as gold nanoparticles, quantum dots, and zirconium ion-immobilized magnetic nanoparticles, have been utilized to improve the sensitivity of PKA activity assays. However, such methods are problematic because limitations are imposed on cost-effectiveness for determining sensitivity and/or kinase activity. Thus, there is a need to develop an assay method for evaluating kinase activity in a manner that is highly sensitive, easy, and economically feasible.

### DISCLOSURE

### Technical Problem

A first object of the present invention is to provide a composition for diagnosing cancer using PKA (Protein Kinase A) activity.

A second object of the present invention is to provide a kit for diagnosing cancer using PKA (Protein Kinase A) activity.

A third object of the present invention is to provide a method of providing information for cancer diagnosis using the above composition or kit.

A fourth object of the present invention is to provide a method of screening a cancer therapeutic agent using the above composition or kit.

### Technical Solution

The present invention provides a composition for cancer diagnosis, comprising an agent for measuring PKA (Protein Kinase A) activity.

The present invention provides a kit for cancer diagnosis, comprising the aforementioned composition.

The present invention provides a method of providing information for cancer diagnosis, using the aforementioned composition or kit.

The present invention provides a method of screening a cancer therapeutic agent using the aforementioned composition or kit.

### Advantageous Effects

According to the present invention, a composition for cancer diagnosis and a kit for cancer diagnosis are used, whereby cancer can be diagnosed in a manner that is highly sensitive, easy, and economically feasible, and a cancer therapeutic agent can be rapidly and accurately screened.

### DESCRIPTION OF DRAWINGS

FIG. 1A schematically shows an on-chip PKA activity assay (GMBS, N-[γ-maleimidobutyryloxy] sulfosuccinimide ester; Ser, serine; Cys, cysteine);

FIGS. 1B to 1E show the test results for optimizing on-chip PKA activity assays, in which FIGS. 1B to 1D illustrate the results of PKA activity based on the fluorescence intensity of array spots, measured after the reaction mixture, comprising kemptide (b), MgCl₂ (c) and ATP (d) at various concentrations and 100 U/mL human cPKA in the reaction buffer, is applied on a well-type peptide array and then incubated for a predetermined period of time, and FIG. 1E illustrates the results of PKA activity based on the fluorescence intensity of array spots, measured after 1 μL aliquots of the reaction mixture including 0.5 mmol/L MgCl₂, 0.5 mmol/L ATP, and 100 U/mL human cPKA are applied on a peptide array and then incubated for a predetermined period of time, the results being expressed as an average of three independent test values±SD;

FIGS. 2A-2F show the high sensitivity of an on-chip PKA activity assay by triton X-100 and an inhibition assay using PKI, in which FIGS. 2A to 2C illustrate the results of PKA activity, measured after the reaction mixture containing triton X-100 and 100 U/mL human cPKA at predetermined concentrations is loaded on a peptide array at 30° C. for 90 min, and specifically, FIG. 2A is a graph showing the dose-dependent increase in sensitivity of on-chip PKA activity assay by triton X-100, FIG. 2B is a graph showing the dose-dependent increase of PKA activity of human cPKA in the presence or absence of 0.01% triton X-100, FIG. 2C is a graph showing the limit of detection (LOD), FIG. 2D illustrates the test results of inter-array reproducibility in the measurement of PKA activity according to the present invention, FIG. 2E illustrates the test results of inter-spot reproducibility in the measurement of PKA activity according to the present invention, and FIG. 2F is a graph showing the dose-dependent inhibition of PKA activity by PKI, wherein the reaction mixture including PKI at a predetermined concentration is applied on the peptide array in the presence of 100 U/mL human cPKA and the PKA activity is then measured and represented as a percentage, the results being expressed as the average of three independent test values±SD;

FIGS. 3A-3C show the results of measurement of sPKA activity of human sera from normal individuals and cancer patients, in which the reaction mixture, including human sera (diluted 20-fold, n=150) from normal individuals (n=30) and hepatic cancer patients (n=30), gastric cancer patients (n=30), lung cancer patients (n=30), and colorectal cancer patients (n=30), is applied on a peptide array and the sPKA activity of 150 serum samples is determined using a standard curve, and specifically, FIG. 3A illustrates representative fluorescence array images, FIG. 3B illustrates the standard curve (r²=0.99) made from the array images of FIG. 3A, and FIG. 3C is a graph showing the sPKA activity distribution in box plots, each box representing the upper and lower quartiles of sPKA activity and the horizontal line of each box showing the median;

FIGS. 4A-4B show the ROC plots of sPKA activity for a serological cancer marker, in which FIG. 4A illustrates the ROC curves of the AUC, sensitivity and specificity of sPKA for each kind of cancer after ROC analysis of hepatic cancer patients (n=30), gastric cancer patients (n=30), lung cancer patients (n=30), and colorectal cancer patients (n=30), and FIG. 4B illustrates the ROC curves of cancer patients (n=120) from which AUC and cut-off values are measured to be 0.966 and 3.5 U/mL, respectively;

FIGS. 5A-5E show the optimization of sPKA autoantibody assays using a cPKA protein array, in which FIG. 5A schematically illustrates an sPKA autoantibody assay, FIG. 5B illustrates results in which human cPKA at a predetermined concentration is applied on an amine-modified array and the binding thereof to rabbit anti-human cPKA is analyzed using alexa546-conjugated anti-rabbit IgG, FIG. 5C is a graph showing the improved binding of anti-human cPKA, achieved by activating human cPKA, wherein 50 μg/mL human cPKA is pre-incubated with PBS (non-activated) or an activity assay buffer (activated) and then applied on the well-type amine array for 60 min, after which the array is incubated with rabbit anti-human cPKA at a predetermined concentration and probed with alexa546-conjugated anti-rabbit IgG, the results being expressed as the average of three independent test values±SD, FIG. 5D illustrates the results of testing of inter-array reproducibility in the measurement of an sPKA autoantibody level according to the present invention, and FIG. 5E illustrates the results of testing of inter-spot reproducibility in the measurement of PKA activity according to the present invention; and

FIGS. 6A-6D show the results of a serological PKA autoantibody assay in human serum from normal individuals and four kinds of cancer patients, in which human serum (diluted 20-fold) from normal individuals (n=30), as well as hepatic cancer patients (n=30), gastric cancer patients (n=30), lung cancer patients (n=30) and colorectal cancer patients (n=30), is applied on a human cPKA protein array, and the array obtained to detect the sPKA autoantibody is incubated with an alexa546-conjugated anti-human IgG and analyzed with a fluorescence scanner, FIG. 6A illustrates the fluorescence array images obtained by analyzing sPKA autoantibody levels in human sera from normal individuals (n=30), hepatic cancer patients (n=30), gastric cancer patients (n=30), lung cancer patients (n=30) and colorectal cancer patients (n=30), using a human cPKA protein array, FIG. 6B illustrates the sPKA autoantibody distribution of human sera in box plots, FIG. 6C illustrates the correlation between an sPKA autoantibody and sPKA activity in human serum, and FIG. 6D illustrates the ROC plot of an sPKA autoantibody assay for four kinds of cancer.

### BEST MODE

The present invention pertains to PKA (Protein Kinase A) activity as a cancer biomarker, and particularly to a composition for diagnosing cancer including an agent for measuring PKA activity, a kit for diagnosing cancer, a method of diagnosing cancer using the above composition, a method of providing information for cancer diagnosis and a method of screening a cancer therapeutic agent.

In order to evaluate the activity of sPKA as a cancer biomarker, the present inventors have experimentally ascertained that sPKA activity in cancer patient groups is notably high by comparing sPKA activity in human sera from patients suffering from hepatic cancer, gastric cancer, lung cancer, and colorectal cancer with that of the serum of a normal individual, using an on-chip PKA activity assay method based on an array, thus culminating in the present invention.

The present invention addresses a composition for cancer diagnosis, including an agent for measuring PKA (Protein Kinase A) activity.

The agent for measuring PKA activity is preferably a substrate that reacts with PKA, and may be a protein that is known to be phosphorylated by PKA. In particular, it may include, but is not limited to, at least one selected from among kemptide, RelA (NF-kappa-B p65 subunit), RhoA (ras homolog gene family, member A; Rho family GTPase), and CREB (cAMP response element-binding protein).

The cancer may include, but is not limited to, at least one selected from among hepatic cancer, gastric cancer, lung cancer, colorectal cancer, esophageal cancer, rectal cancer, prostate cancer, melanoma, thyroid cancer, liposarcoma, bladder cancer, ovarian cancer, and renal cancer.

In the present invention, “diagnosis” means that the presence of a pathological condition or a feature thereof is identified. For the purpose of the present invention, diagnosis means that the onset of cancer is identified.

In the present invention, the measurement of PKA (Protein Kinase A) activity may be a process of evaluating the extent of reaction of a substrate with PKA using a substrate that reacts with PKA, and specifically, may be a process of probing the phosphorylation of a substrate caused by PKA. Assay methods therefor may include methods of using an antibody for recognizing a phosphate group, methods of using a chemical for recognizing a phosphate group, methods employing luminescence, etc., but the present invention is not limited thereto.

The chemical for recognizing the phosphate group may be a molecular probe such as a biotinylated phosphate-specific ligand based on a Zn²⁺ complex, and particularly Phos-tag.

Furthermore, ELISA, western blotting, flow cytometry, immunofluorescence, immunohistochemistry or mass spectrometry may be used, but the present invention is not limited thereto.

The composition for cancer diagnosis may further include an additional ingredient, depending on the end use thereof.

In addition, the present invention addresses a kit for cancer diagnosis, including the composition for cancer diagnosis. Preferably, the kit for cancer diagnosis may further include a composition including any one or more kinds of additional ingredients suitable for assay methods, or a solution or device therefor.

For example, as in the testing of the examples of the present invention, the kit for cancer diagnosis may adopt a high-throughput on-chip sPKA activity array. The on-chip sPKA activity array uses a PKA substrate peptide and a small-molecule fluorescent phosphor sensor and exhibits quantitatively high sensitivity, reproducibility, and cost-reduction effects.

In addition, the present invention addresses a method of providing information for cancer diagnosis using the composition for cancer diagnosis or the kit for cancer diagnosis, and preferably includes the following steps of measuring PKA (Protein Kinase A) activity from a biosample separated from a cancer-suspected patient and comparing the PKA activity with the protein level of a normal control sample.

The descriptions of the agent for measuring PKA activity, the substrate reacting with PKA, the cancer, and the measurement of PKA activity remain the same as those of the composition for cancer diagnosis or the kit for cancer diagnosis described above.

The biosample separated from the cancer-suspected patient may be blood or serum, but the present invention is not limited thereto.

In addition, the present invention addresses a method of screening a cancer therapeutic agent using the composition for cancer diagnosis or the kit for cancer diagnosis, and includes the following steps of (a) bringing a sample to be analyzed into contact with a composition including an agent for measuring PKA activity, (b) measuring the PKA activity of the sample, and (c) determining the sample to be a cancer therapeutic agent when the PKA activity is observed to be down-regulated.

The descriptions of the agent for measuring PKA activity, the substrate reacting with PKA, the cancer, and the measurement of PKA activity remain the same as those of the composition for cancer diagnosis or the kit for cancer diagnosis described above.

The sample to be analyzed is an unknown candidate for use in a screening process, in order to evaluate the effect thereof or the potential thereof on the treatment or prevention of cancer or on the inhibition of cancer metastasis. Examples of the sample may include, but are not limited to, chemicals, natural extracts, nucleotides, antisense-RNA and the like.

### MODE FOR INVENTION

A better understanding of the present invention may be obtained via the following examples, which are set forth to illustrate, but are not to be construed as limiting the scope of the present invention.

### REFERENCE EXAMPLE

Chemical Reagent

Used for the present test, 3-aminopropyltrimethoxysilane, BSA, human serum albumin, ATP and H89 were obtained from Sigma-Aldrich (St. Louis, Mo.). Also, PKA peptide inhibitor (PKI) and cPKA were purchased from Biaffin GmbH & Co KG (Kassel, Germany). N-[γ-maleimidobutyloxy]succinimide ester (GMBS) was obtained from Pierce (Rockford, Ill.). As the PKA substrate peptide, kemptide (C-G-G-L-R-R-A-S-L-G), synthesized by Peptron (Daejeon, Korea), was used. A pro-Q diamond phosphoprotein gel stain and a destaining solution were purchased from Invitrogen (Carlsbad, Calif.). A poly(dimethylsiloxane) (PDMS) solution was obtained from Sewang Hitech (Gimpo, Korea).

Serum Sample

Human serum samples (n=30) obtained from normal individuals and samples obtained from hepatic cancer patients (n=30), gastric cancer patients (n=30), lung cancer patients (n=30) and colorectal cancer patients (n=30) were supplied by the Biobank, Kangwon National University Hospital (which is a member of the National Biobank Korea, Korea), and were stored at −80° C. until use. Testing using human samples was performed with the approval of the Ethics Committee of local labs for human research.

Data Analysis

In order to achieve quantification of the fluorescence intensity and data extraction, ScanArray Express software was utilized. The Origin 6.0 software package (Origin Lab, Northampton, Mass.) was used to conduct t-tests on the two groups. A p value of less than 0.05 was regarded as statistically significant. With the goal of calculating AUC (Area Under Curve), sensitivity and specificity, ROC (Receiver Operating Characteristics) analysis was performed using MedCalc statistical software 11.4.4.0 (Mariakerke, Belgium).

### Example 1

### Manufacture of Well-Type Peptide Array and PKA Activity Assay Using the Same

### Example 1-1

### Manufacture of Well-Type Peptide Array

(1) Manufacture of PDMS Gasket

5 g of a PDMS base and 0.5 g of a curing agent were mixed so as to be cloudy with bubbles and then defoamed at room temperature for 30 min, thus preparing a PDMS prepolymer solution. This mixture was poured into a chromium-coated copper mold (Amogreentech, Gimpo, Korea) having an array of poles with a diameter of 1.5 mm and a height of 0.3 mm. The mold was incubated at 84° C. for 90 min, after which a PDMS gasket having an array of wells having a diameter of 1.5 mm was separated therefrom and was then stored on a transparent film until use.

(2) Manufacture of Well-Type Peptide Array

According to a known method (Jung J W, Jung S H, Yoo J O, Suh I B, Kim Y M, Ha K S. Label-free and quantitative analysis of C-reactive protein in human sera by tagged-internal standard assay on antibody arrays. Biosens Bioelectron 2009; 24:35 1469-73.), an amine-modified glass slide was prepared. Specifically, a glass slide (75×25 mm) was washed with H₂O₂/NH₄OH/H₂O (1:1:5, v/v) at 70° C. for 10 min. The slide was immersed for 2 hr in a 95% ethanol solution containing 1.5% 3-aminopropyltrimethoxiysilane (v/v) and fired at 110° C.

The PDMS gasket was mounted on the amine-modified glass slide to manufacture a well-type amine array. The amine array was sequentially modified with 5 mmol/L sulfo-GMBS in a 50 mmol/L sodium bicarbonate buffer (pH 7.0) and a 10 μg/mL substrate peptide (8.1 mmol/L Na₂HPO₄, 1.2 mmol/L KH₂PO₄, pH 7.4) in a phosphate buffer, thereby yielding a well-type amine array. The N-hydroxysuccinimidyl ester and the maleimide moiety of sulfo-GMBS were bound to the amine-modified glass surface of the array and the cysteine residue of the substrate peptide, respectively.

### Example 1-2

### PKA Activity Assay Using Well-Type Peptide Array

(1) On-Chip PKA Activity Assay Using Peptide Array Based on Fluorescence

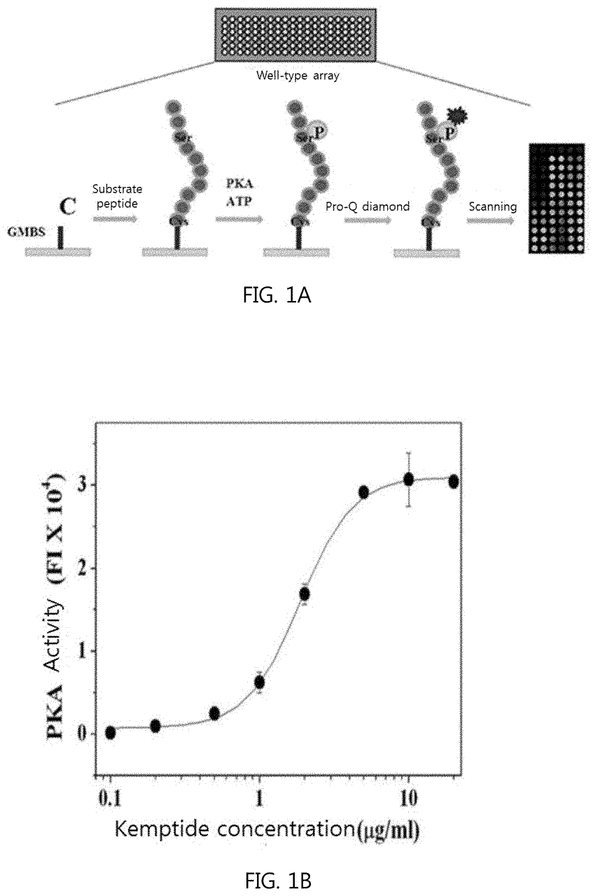

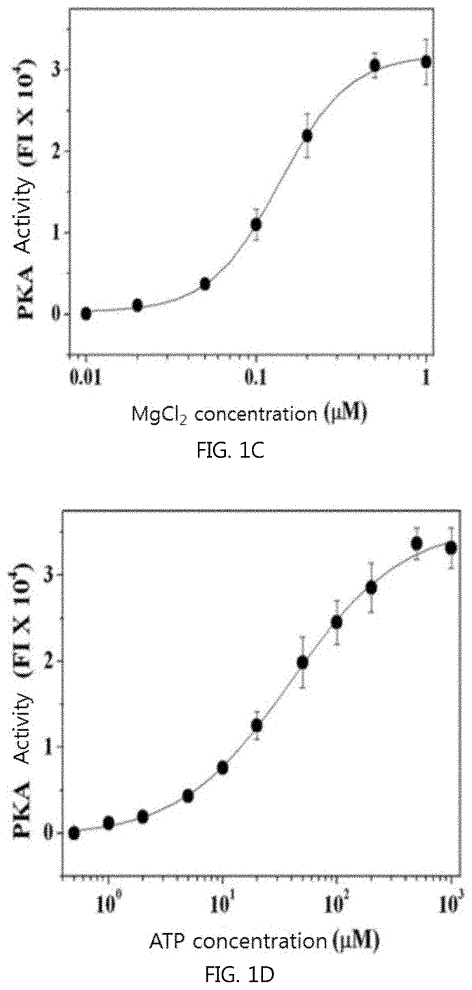

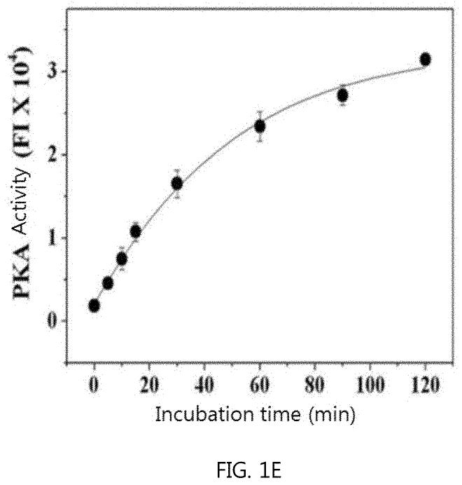

FIG. 1A schematically shows the on-chip PKA activity assay (GMBS, N-[γ-maleimidobutyryloxy]sulfosuccinimide ester; Ser, serine; Cys, cysteine). As shown in FIG. 1A, a PKA activity assay was performed on the well-type peptide array using a pro-Q diamond stain.

Specifically, the peptide array was blocked at 37° C. for 30 min with 1% BSA in TBS (13.8 mmol/L NaCl and 2 mmol/L Tris-HCl, pH 7.4) containing 0.1% Tween-20, and was then sequentially washed with TBS containing 0.1% Tween-20 and Milli-Q water. 1 μL of a reaction mixture, comprising an activity assay buffer (50 mmol/L Tris-HCl, pH 7.5, 0.5 mmol/L MgCl₂, 0.01% Triton X-100, 500 μmol/L ATP and 0.2% human serum albumin) and diluted serum (20-fold), was applied on the peptide array, both in the presence and in the absence of 2 μmol/L PKI, and then incubated at 30° C. for 90 min. Meanwhile, in order to manufacture a standard curve for quantitative measurement of PKA activity, the reaction mixture containing cPKA at various concentrations was applied on the peptide array.

The array was incubated at room temperature for 60 min together with a pro-Q diamond stain to probe the phosphorylated serine residue of the peptide substrate. The array was washed two times with a destaining solution for 15 min and was then washed two times with Milli-Q water for 5 min. The resulting array was scanned by means of a fluorescence scanner (ScanArray Express GX, Perkin Elmer, Waltham, Mass.) using a laser at 543 nm, and the fluorescence intensity of the array spots thus measured was used to determine PKA activity.

(2) Determination of PKA Activity

For quantitative measurement of PKA activity, a standard curve consisting of a linear fit was made using the Origin program:

y=ax+b

in the above equation, y is the fluorescence intensity of a sample on the surface of an array, a and b are the slope and the intercept of the linear fit of the standard curve, respectively, and x is the PKA activity. The PKA activity in the serum sample is calculated from the difference in PKA activity between the absence of PKI and the presence of PKI, and is represented in U/mL.

(3) Optimization of On-Chip sPKA Activity Assay Using Peptide Array Based on Fluorescence

As shown in FIG. 1A, a high-sensitivity quantitative assay was performed in order to analyze sPKA activity in human serum samples using a pro-Q diamond phosphor-sensor.

In order to optimize PKA activity assay, the reaction mixture, comprising kemptide, MgCl₂ and ATP at various concentrations, was applied on a GMBS-modified well-type array. Specifically, kemptide, MgCl₂ and ATP at various concentrations were mixed with 100 U/mL human cPKA in the reaction buffer to prepare the reaction mixture, which was then applied on the well-type peptide array. Also, 1 μL of the reaction mixture, comprising 10 μg/mL peptide, 0.5 mmol/L MgCl₂, 0.5 mmol/L ATP, and 100 U/mL human cPKA, was applied on the peptide array and incubated for a predetermined period of time.

The fluorescence intensity of the array spots was measured using the aforementioned method to determine PKA activity. The results are expressed as the average of three independent test values±SD, and are shown in FIGS. 1B to 1E. As shown in FIG. 1B, the PKA activity, represented by the fluorescence intensity, was increased in a concentration-dependent manner by kemptide, and exhibited maximal effects at 10 μg/mL. MgCl₂ also increased the PKA activity in a dose-dependent manner, and was saturated at 0.5 mmol/L (FIG. 1C). ATP increased the PKA activity in a dose-dependent manner, and exhibited maximal stimulation at 0.5 mmol/L (FIG. 1D). The PKA activity was also increased in a time-dependent manner (FIG. 1E).

(4) Increase in Sensitivity of On-Chip PKA Activity Assay by Triton X-100 and Characterization of PKA Activity Assay

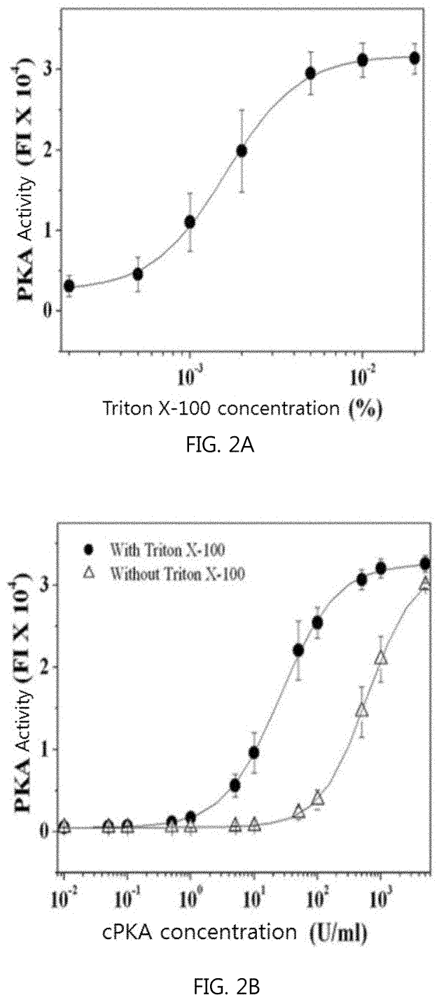

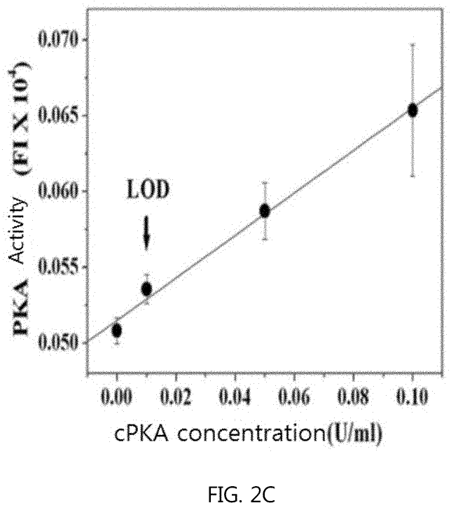

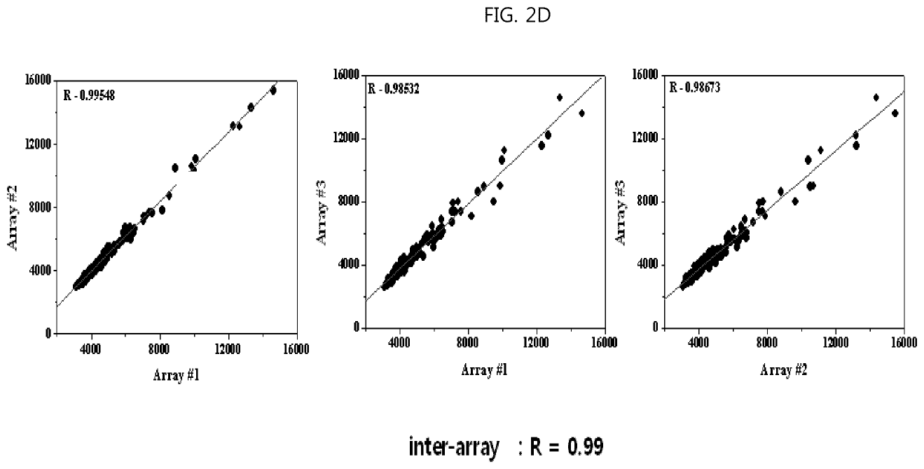

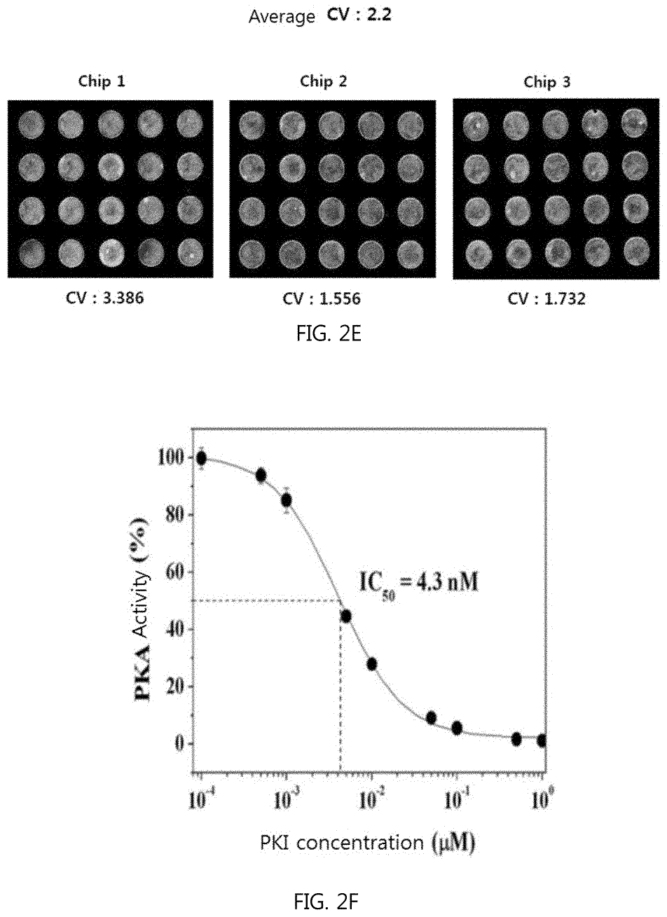

Whether triton X-100 was able to increase the sensitivity of an on-chip PKA activity assay was tested. FIGS. 2A to 2C show the results of measurement of PKA activity after the reaction mixture containing triton X-100 and 100 U/mL human cPKA at predetermined concentrations was loaded on the peptide array at 30° C. for 90 min. As shown in FIG. 2A, triton X-100 increased the PKA activity in a dose-dependent manner, manifested apparent activation at 0.001%, and was saturated at 0.01%. Then, in the presence or absence of triton X-100, the PKA activity of the reaction mixture, containing cPKA at a predetermined concentration, was analyzed, and the effects of triton X-100 on the sensitivity of the PKA activity assay were measured (FIGS. 2B and 2C). Triton X-100 promoted a dose-dependent increase in PKA activity. The limit of detection of the PKA activity assay was increased by 0.01 U/mL from 1.45 using 0.01% triton X-100, from which the PKA activity assay was found to be enhanced by triton X-100.

Using human sera (n=150), inter-array reproducibility and inter-spot reproducibility were analyzed to thus evaluate the reproducibility of the on-chip PKA activity assay.

The inter-array reproducibility was determined by analyzing the reaction mixture at the same position on different arrays. The test results are shown in FIG. 2D. As shown in FIG. 2D, the average correlation coefficient was 0.990 (n=3, CV=0.7%), and thus inter-array reproducibility was evaluated to be high. Also, the inter-spot reproducibility was determined by analyzing 20 overlapping spots. The results are shown in FIG. 2E. The average coefficient of variation was 2.2% (n=3). In brief, these results show that the on-chip PKA activity assay exhibits high reproducibility.

Next, the inhibitory effects on the on-chip PKA activity assay of PKI as the PKA-specific inhibitor were analyzed. FIG. 2F is a graph showing the dose-dependent inhibition of PKA activity by PKI. In the presence of 100 U/mL human cPKA, the reaction mixture containing PKI at a predetermined concentration was applied on the peptide array, and the PKA activity was represented as a percentage. The results are expressed as the average of three independent test values±SD. As shown in FIG. 2F, when 100 U/ml cPKA was used, PKI inhibited PKA activity in a dose-dependent manner, and the maximal effect thereof was exhibited at 0.5 μM. The IC₅₀ (half-maximum inhibitory concentration) of PKI on PKA activity assay was calculated to be 4.3 nM. Thereby, specific sPKA activity in the human blood sample can be determined using PKI and the on-chip PKA activity assay is evaluated as being appropriate for screening the PKA inhibitor.

(5) Measurement of sPKA Activity of Human Sera from Normal Individuals and Patients with Four Kinds of Cancer

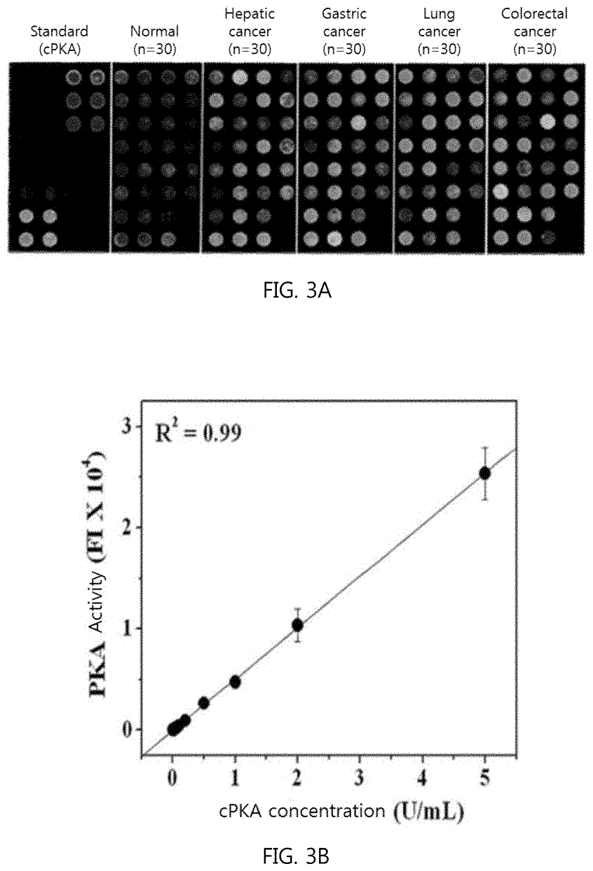

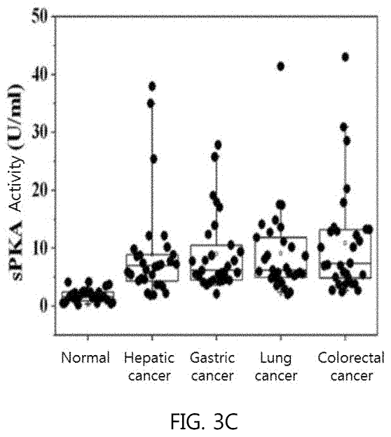

In order to evaluate the activity of sPKA as a cancer biomarker, the following test was performed. Specifically, an on-chip activity assay was performed to determine the sPKA activity of human sera from normal individuals (n=30) and hepatic cancer patients (n=30), gastric cancer patients (n=30), lung cancer patients (n=30) and colorectal cancer patients (n=30). The reaction mixture comprising cPKA at various concentrations and diluted human serum was applied on the peptide array, and phosphorylated serine of kemptide was probed with a pro-Q diamond stain. The sPKA activity was measured using the standard curve made based on the fluorescence intensity of cPKA (FIGS. 3A and 3B). FIG. 3A illustrates the typical fluorescence array images, and FIG. 3B illustrates the standard curve made from the array images of FIG. 3A (r²=0.99).

In order to exclude the non-specific signal of anode kinase, sPKA activity in the presence of PKI was subtracted from sPKA activity in the absence of 0.5 μM PKI to thereby determine human serum sPKA activity. The results are shown in the box plots (FIG. 3C). FIG. 3C is a graph showing the sPKA activity distribution in box plots. Each box represents the upper and lower quartiles of sPKA activity. The horizontal line of each box indicates the median. The average sPKA activity values of normal individuals and patients with hepatic cancer, gastric cancer, lung cancer and colorectal cancer were 1.78±1.09, 8.92±8.72, 8.96±6.53, 9.06±7.50 and 10.94±9.34 U/mL, respectively, and the sPKA activity in all cancer patients was much higher than in normal individuals (p<10⁻⁴). Therefore, the on-chip PKA activity assay is suitable for determining the sPKA activity of human serum, and the sPKA activity can be used as a biomarker for cancer diagnosis.

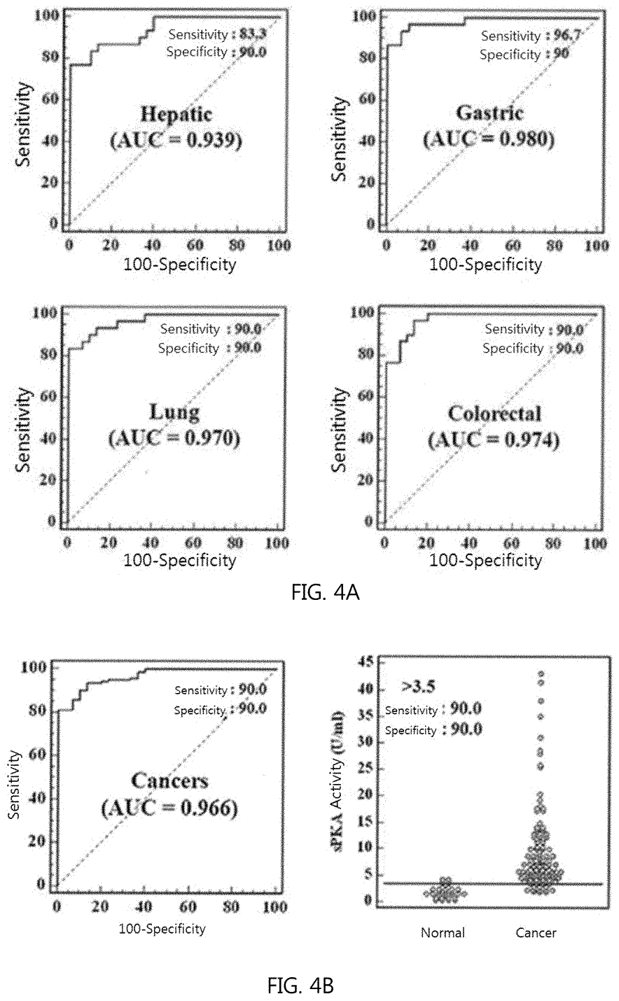

In order to evaluate the activity of sPKA as a cancer biomarker, an on-chip activity assay was performed for four kinds of cancer as described above, and ROC analysis was conducted (FIG. 4A). FIG. 4A shows the ROC curves of AUC, sensitivity and specificity of sPKA for each kind of cancer after the ROC analysis of hepatic cancer patients (n=30), gastric cancer patients (n=30), lung cancer patients (n=30), and colorectal cancer patients (n=30). The results of sensitivity and specificity of sPKA activity obtained for the four kinds of cancer are as follows: hepatic cancer (83.3% and 90.0%), gastric cancer (96.7% and 90%), lung cancer (90.0% and 90.0%), and colorectal cancer (90.0% and 90.0%).

The following high AUC values were obtained: 0.939 (95% confidence interval, 0.85-0.98), 0.980 (95% confidence interval, 0.91-0.99), 0.970 (95% confidence interval, 0.89-0.99) and 0.974 (95% confidence interval, 0.90-0.99).

In FIG. 4B, AUC and cut-off values are measured to be 0.966 and 3.5 U/mL, respectively, from the ROC curve of cancer patients (n=120). The sPKA activity assay of all of the cancer patients exhibited a sensitivity of 90.0% and a specificity of 90.0%, and in particular, an AUC value of 0.966 (95% confidence interval, 0.92-0.98) and a cut-off value of 3.5 U/mL were obtained, which are evaluated to be higher than conventionally reported values. Hence, sPKA activity is regarded as a potential biomarker for cancer diagnosis.

Consequently, sPKA activity in human sera of patients with hepatic cancer, gastric cancer, lung cancer and colorectal cancer was much higher than that of the control group. However, there was no significant difference in sPKA autoantibody between the cancer group and the normal group. Furthermore, in human sera, sPKA activity was observed to have no correlation with the sPKA autoantibody level. Thus, sPKA activity, rather than the sPKA autoantibody, is deemed to be suitable for use as a biomarker for cancer diagnosis. Also, the on-chip sPKA activity assay is effective for cancer diagnosis, and has very high potential for use in inhibitor screening and in the diagnosis of PKA-related human diseases.

### Comparative Example 1

### Manufacture of cPKA Protein Array and Analysis of Human Serum sPKA Autoantibody Level Using the Same

(1) Manufacture of Human cPKA Protein Array and sPKA Autoantibody Assay in Human Serum Sample

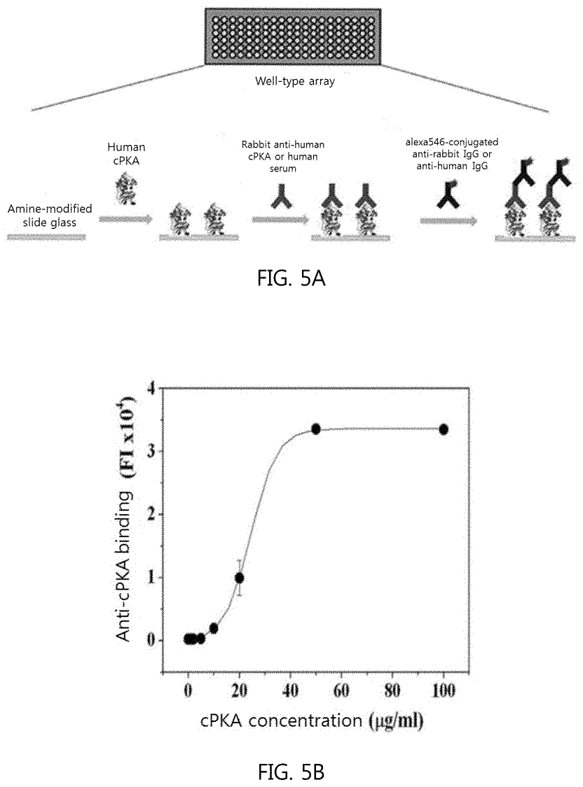

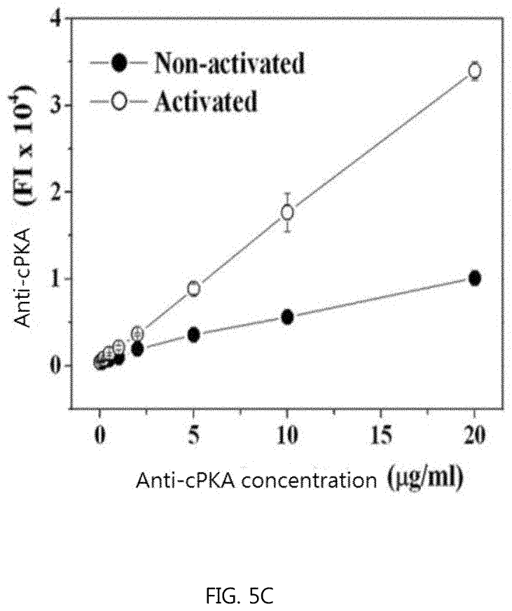

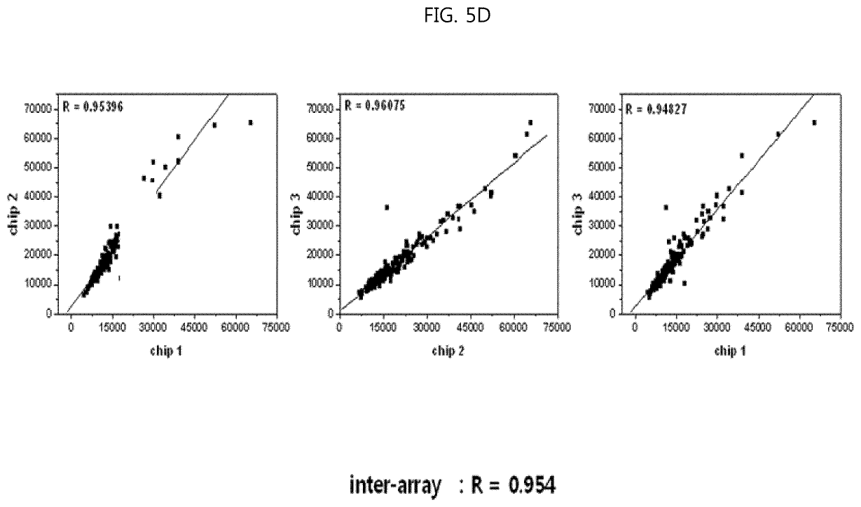

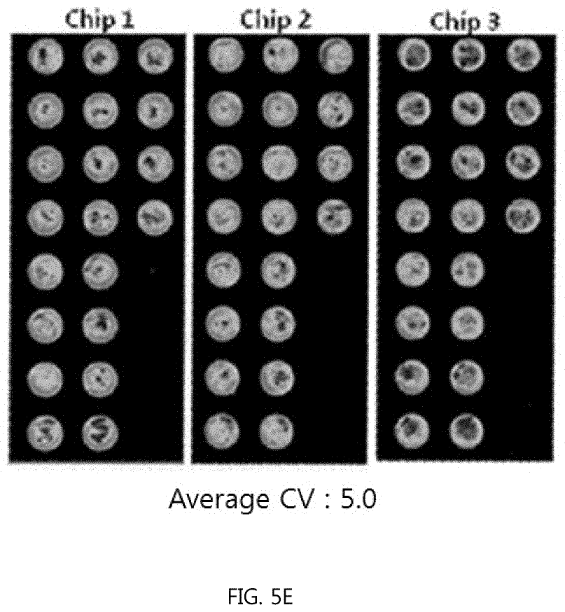

FIG. 5A schematically shows the sPKA autoantibody assay. As shown in FIG. 5A, the human serum sPKA autoantibody level was analyzed using a cPKA protein array. To manufacture the human cPKA protein array, human cPKA was prepared at various concentrations in PBS (8.1 mmol/L Na₂HPO₄, 1.2 mmol/L KH₂PO₄, pH 7.4, 2.7 mmol/L KCl, and 138 mmol/L NaCl; non-activated) on ice or in an activity assay buffer (activated) and then applied on the well-type amine array at 37° C. for 60 min. The array thus obtained was sequentially washed with PBS containing 0.1% Tween-20 (PBST) for 10 min, and with Milli-Q water for 5 min. The array was blocked at 37° C. for 60 min using 1% BSA in PBST. Rabbit anti-human PKA in PBS containing 0.05% Tween-20 or 20-fold diluted human serum was applied at a predetermined concentration on the human cPKA array at 37° C. for 60 min, and then probed at 37° C. for 60 min using 10 g/mL alexa546-conjugated anti-rabbit IgG or anti-human IgG in PBS containing 0.05% Tween-20 and 1% BSA. The array was washed with PBST for 10 min, further washed with Milli-Q water for 5 min, and dried in air. Thereafter, the array was scanned by means of a fluorescence scanner using a laser at 543 nm.

(2) Optimization of Serological PKA Autoantibody Assay using cPKA Protein Array

In order to optimize the sPKA autoantibody assay, as shown in FIG. 5A, human cPKA was immobilized at a predetermined concentration on the surface of the well-type amine array, thus manufacturing a protein array, and a reaction mixture including rabbit anti-human cPKA was applied on the cPKA protein array, and the binding thereof to rabbit anti-human cPKA was analyzed through probing with alexa546-conjugated anti-rabbit IgG. FIG. 5B shows the results, in which human cPKA was applied at a predetermined concentration on the amine-modified array and the binding thereof to rabbit anti-human cPKA was analyzed with alexa546-conjugated anti-rabbit IgG. As shown in FIG. 5B, human cPKA increased the binding of anti-human cPKA in a dose-dependent manner, and was saturated at 50 μg/mL.

Next, whether the activation of human cPKA with the activity assay buffer was able to increase the binding affinity of anti-human cPKA to cPKA was tested. Human cPKA was pre-incubated with PBS (non-activated) or an activity assay buffer (activated), and was then immobilized onto the well-type amine array, and the binding of anti-human cPKA was analyzed. FIG. 5C is a graph showing the improved binding of anti-human cPKA, achieved by activating human cPKA. As shown in FIG. 5C, the activation of cPKA significantly increased the binding of anti-human cPKA to its antigen. Also, under both of the above two conditions, the binding of anti-human cPKA and human cPKA protein array was linearly proportional up to an antibody concentration of 40 μg/mL.

In order to measure the reproducibility of the on-chip PKA assay, inter-array reproducibility and inter-spot reproducibility were tested using the same procedures, and the on-chip sPKA autoantibody assay reproducibility was evaluated using human sera (n=150). The results are shown in FIG. 5D. Based on the test results, the average value (n=3, CV=0.7%) of the correlation coefficient was 0.954, from which inter-array reproducibility was evaluated to be high. Furthermore, the results of evaluation of inter-spot reproducibility are shown in FIG. 5E, where it is evaluated based on a coefficient of variation of 5.0% (n=3). Therefore, the sPKA autoantibody assay using the human cPKA protein array is suitable for use in anti-cPKA assays on human sera.

(3) Analysis of Correlation Between sPKA Autoantibody Level in Human Serum Sample and sPKA Activity

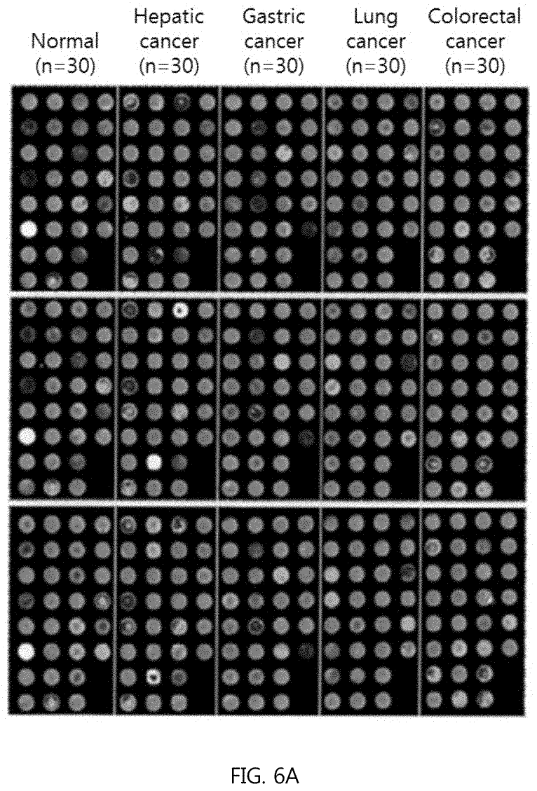

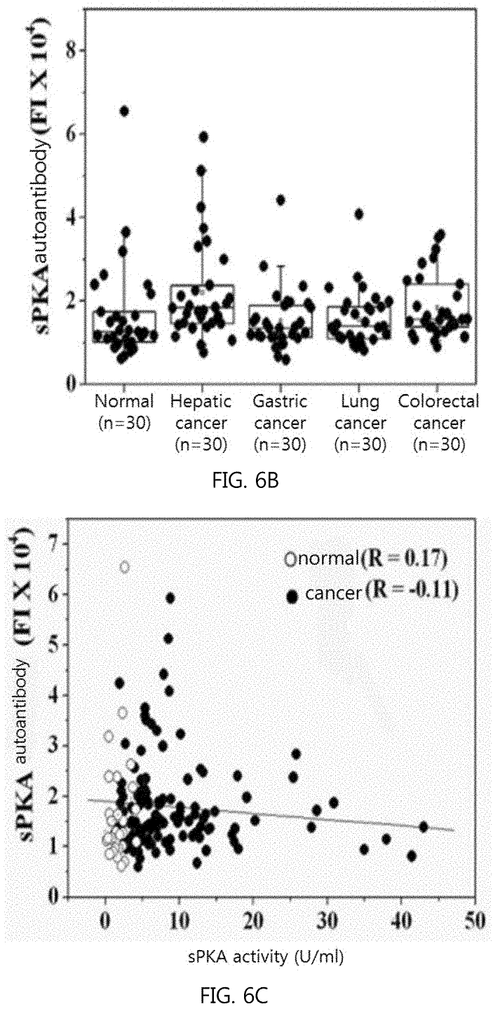

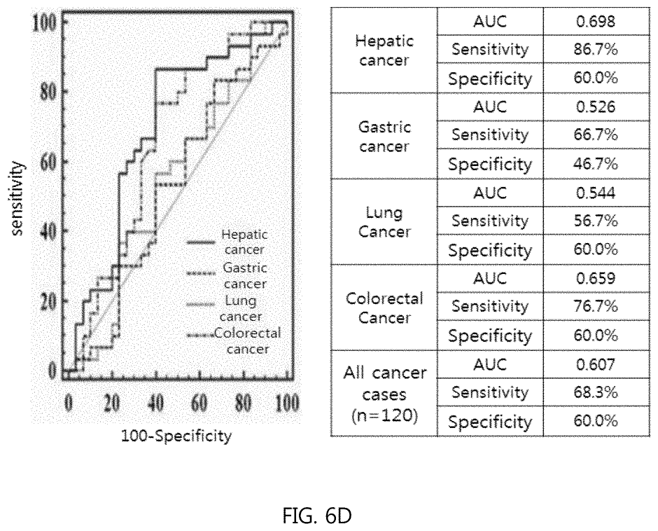

In order to evaluate the PKA autoantibody as the cancer biomarker, as shown in FIG. 5A, the sPKA autoantibody level in human sera from a normal group (n=30) and a patient group comprising hepatic cancer patients (n=30), gastric cancer patients (n=30), lung cancer patients (n=30) and colorectal cancer patients (n=30) was measured using the human cPKA protein array (FIG. 6A), and was utilized in an sPKA activity assay. FIG. 6B shows the sPKA autoantibody distribution of human sera in box plots. As shown in FIG. 6B, there was no significant difference in PKA autoantibody level between the normal group and the four cancer groups (p>0.05), which means that the PKA autoantibody level is not a good biomarker for cancer diagnosis.

Additionally, the correlation coefficient between PKA autoantibody level and sPKA activity in the sera of normal cells and cancer patients was measured. FIG. 6C shows the correlation between sPKA autoantibody and sPKA activity in human sera. The sPKA activity distribution had no relationship with the PKA autoantibody level distribution, and R values for the normal individuals and the cancer patients were respectively 0.17 and −0.11 (FIG. 6C). FIG. 6D shows the ROC plot of an sPKA autoantibody assay for four kinds of cancer. The AUC, sensitivity and specificity values for each kind of cancer are given in the table. The AUC values of the sPKA autoantibody were 0.698 (hepatic, sensitivity: 86.7%, specificity: 60.0%), 0.526 (gastric, sensitivity: 66.7%, specificity: 46.7%), 0.544 (lung, sensitivity: 56.7%, specificity: 60.0%), and 0.659 (colorectal, sensitivity: 76.6%, specificity: 60.0%), which were observed to be much lower than the values of sPKA activity (FIG. 6D). Also, all of the cancer patients (n=120) exhibited AUC of 0.607, a sensitivity of 68.3%, and a specificity of 60.0%, which were evaluated to be much lower than the values of sPKA activity (FIG. 6D).

Based on these results, the sPKA autoantibody level is not regarded as a good biomarker for cancer diagnosis, compared to sPKA activity.

Also, compared to the method of Comparative Example 1 for analyzing the protein kinase through autoantibody assay, the method of measuring the protein kinase activity through phosphorylation of the substrate that reacts with the protein kinase, according to the present invention, has been found to measure protein kinase activity in a manner that is highly sensitive, easy, and economically feasible.

### INDUSTRIAL APPLICABILITY

According to the present invention, a composition for cancer diagnosis and a kit for cancer diagnosis are used, whereby cancer can be diagnosed in a manner that is highly sensitive, easy, and economically feasible, and a cancer therapeutic agent can be rapidly and accurately screened.

What is claimed is:

1. A kit for diagnosing cancer, comprising kemptide as an agent for measuring PKA (Protein Kinase A) activity, a glass surface, and GMBS (N-[γ-maleimidobutyryloxy]sulfosuccinimide ester) as a linker for connecting the glass surface and the kemptide.

2. The kit of claim 1, wherein the agent for measuring PKA activity is a substrate that reacts with PKA.

3. The kit of claim 1, wherein PDMS (poly(dimethylsiloxane)) gasket is mounted on the glass surface.

4. The kit of claim 1, wherein the cancer is at least one selected from among the group consisting of hepatic cancer, gastric cancer, lung cancer, colorectal cancer, esophageal cancer, rectal cancer, prostate cancer, melanoma, and thyroid cancer.

5. The kit of claim 1, further comprising Triton X-100.
